(19) United States
(12) Patent Application Publication
(10) Pub. No.: US 2026/0069649 A1
(43) Pub. Date: Mar. 12, 2026

(54) COMPOSITION COMPRISING FERMENTED MORINDA CITRIFOLIA EXTRACT HAVING OSTEOARTHRITIS ALLEVIATION EFFECT

(71) Applicant: NSTBIO CO., LTD., Gimpo-si (KR)

(72) Inventors: Eunyoung PARK, Incheon (KR); Jaeyeon LEE, Suwon-si (KR); Eunmin KIM, Incheon (KR); Jisoo CHOI, Incheon (KR); Heeyeon KWON, Seoul (KR)

(73) Assignee: NSTBIO CO., LTD., Gimpo-si (KR)

(21) Appl. No.: 19/390,170

(22) Filed: Nov. 14, 2025

Related U.S. Application Data
(63) Continuation of application No. PCT/KR2024/006640, filed on May 16, 2024.

(30) Foreign Application Priority Data
May 15, 2023 (KR) 10-2023-0062383
May 16, 2024 (KR) 10-2024-0063567

(51) Int. Cl. A61K 36/746; A61P 19/02

(52) U.S. Cl. CPC A61K 36/746; A61P 19/02; A61K 2236/15; A61K 2236/19; A61K 2236/51

(57) ABSTRACT

The present invention relates to a composition comprising a fermented Morinda citrifoliaextract and having an osteoarthritis alleviation effect, and a method for preparing the composition comprising the fermented Morinda citrifolia extract. The present invention provides a composition comprising a fermented Morinda citrifolia extract and having an osteoarthritis alleviation effect.

### CROSS-REFERENCE TO RELATED APPLICATION

This application is a continuation of the U.S. National Stage of International Application No. PCT/KR2024/006640 filed May 16, 2024, which claims the benefit of priority from the prior Korean Patent Application No. 10-2023-0062383, filed on May 15, 2023, and Korean Patent Application No. 10-2024-0063567, filed on May 16, 2024, the disclosures of which are incorporated herein by reference in its entirety.

### TECHNICAL FIELD

The present invention relates to a composition including a fermented Morinda citrifolia extract (FME) having an osteoarthritis alleviation effect and a method of preparing a composition including an FME.

### BACKGROUND ART

Degenerative arthritis primarily occurs in weight-bearing joints like the knees and spine. The only fundamental treatment is surgery, and other options include anti-inflammatory medications for pain relief.

Acetaminophen and nonsteroidal anti-inflammatory drugs (NSAIDs) are used to relieve arthritis pain, but long-term use thereof may cause digestive and blood clotting problems and have side effects such as nausea, vomiting, constipation, loss of appetite, and dizziness.

Hyaluronic acid and glucosamine, as cartilage protectors, may help slow down joint degeneration, but their effectiveness in promoting cartilage production and inducing regeneration is limited.

To date, no medication has been developed to prevent or treat degenerative arthritis. While there are various notified ingredients and individually recognized ingredients that are beneficial for joint health, the development of new materials is still necessary.

Morinda citrifolia has anti-inflammatory, anti-cancer, and anti-bacterial effects and is effective in diabetes and cardiovascular diseases, and the iridoid substance contained in Morinda citrifolia has also been reported to have antioxidant, anti-bacterial, liver function improvement, and heart function improvement effects. Fermented Morinda citrifolia extract (FME) obtained through fermentation technology is an extract with increased iridoid components, and previous research results have shown that FME has an improvement effect on inflammatory cytokines. Therefore, the present inventors attempted to confirm the osteoarthritis alleviation effect of FME using an osteoarthritis in vitro model in which an inflammatory response was induced by IL-1β treatment in SW1353 cells, a human chondrosarcoma cell line.

### DISCLOSURE

### Technical Problem

The present invention is directed to providing a composition including a fermented Morinda citrifolia extract (FME) having an osteoarthritis alleviation effect and a method of preparing the same.

### Technical Solution

To achieve the above-described object, the present invention provides a composition including a fermented Morinda citrifolia extract (FME) having an osteoarthritis alleviation effect.

In one embodiment, the FME may contain deacetylasperulosidic acid as an active ingredient.

In one embodiment, the active ingredient may be contained in an amount of 5.76 to 8.64 mg per 1 g of the FME.

In one embodiment, the FME may be prepared by inoculating Morinda citrifolia with Lactobacillus plantarum NST1805 (Accession Number: KCCM12833P) and then fermenting it.

In one embodiment, the FME may be prepared by juicing the fermented Morinda citrifolia and concentrating the juice to 55 to 65 Brix.

In addition, the present invention provides a food composition having an osteoarthritis alleviation effect, including FME.

In addition, the present invention provides a method of preparing a composition including a fermented Morinda citrifolia extract having an osteoarthritis alleviation effect, including: inoculating Morinda citrifolia with lactic acid bacteria and fermenting it; preparing a fermented Morinda citrifolia extract from the fermented Morinda citrifolia obtained by fermentation; and concentrating the fermented Morinda citrifolia extract.

### Advantageous Effects

According to the present invention, the fermented Morinda citrifolia extract (FME) has an anti-inflammatory effect and a cartilage decomposition protein inhibition effect on osteoarthritis.

### DESCRIPTION OF DRAWINGS

FIG. 1 is a graph illustrating the effect of the fermented Morinda citrifolia extract (FME) according to the present invention on nitric oxide (NO) production.

FIGS. 2A, 2B, 3A, 3B, and 4 are graphs illustrating the inhibitory effect of the FME according to the present invention on cartilage degradation and inflammation-related protein expression.

FIGS. 5 and 6 are graphs illustrating the inhibitory effect of the FME according to the present invention on cartilage degradation and inflammation-related mRNA expression.

FIG. 7 is a graph illustrating the MMP-9 inhibitory effect of FME according to the present invention.

### MODES OF THE INVENTION

Hereinafter, embodiments disclosed herein will be described in detail with reference to the attached drawings. Regardless of the drawing numbers, identical or similar components will be given the same reference numerals and redundant descriptions thereof will be omitted. In addition, when describing embodiments disclosed herein, when it is determined that a detailed description of related known technology may obscure the gist of the embodiments disclosed herein, the detailed description thereof will be omitted. In addition, the attached drawings are only intended to facilitate easy understanding of the embodiments disclosed herein, and the technical ideas disclosed herein are not limited by the attached drawings, and they should be understood to include all modifications, equivalents, and substitutes included in the spirit and technical scope of the present invention.

Terms that include ordinal numbers such as first and second may be used to describe various components, but the components are not limited by these terms. These terms are used solely to distinguish one component from another.

Singular forms include plural forms, unless the context clearly indicates otherwise.

Terms used herein, such as “comprise” or “have,” are intended to specify the presence of features, numbers, steps, operations, components, or combinations thereof described herein and should not be understood as precluding the possibility of the presence or addition of one or more other features, numbers, steps, operations, components, or combinations thereof.

The present invention relates to a composition including a fermented Morinda citrifolia extract (FME) having an osteoarthritis alleviation effect and a method of preparing a composition including FME.

The term ‘fermented Morinda citrifolia’ herein refers to Morinda citrifolia inoculated with lactic acid bacteria and then fermented and aged for a specified period of time.

The term ‘FME’ herein refers to a liquid obtained by performing at least one concentration process on the juice of the fermented Morinda citrifolia.

The term ‘composition having an osteoarthritis alleviation effect’ herein refers to a group of food to which added value is given by using physical, biochemical, or bioengineering techniques for the food so that the function of the food can be performed and expressed for a specific purpose, or a composition that has been designed and processed to sufficiently express the body's regulatory function of food composition for regulating the biological defense rhythm, disease prevention, and recovery, and the like.

Herein, compositions having an osteoarthritis alleviation effect include various food, beverages, gum, tea, vitamin complexes, functional foods, and the like. Furthermore, the present invention may contain various nutrients, vitamins, minerals (electrolytes), flavoring agents such as synthetic flavoring agents and natural flavoring agents, coloring agents and fillers, pectic acid and salts thereof, alginic acid and salts thereof, organic acids, protective colloid thickeners, pH adjusters, stabilizers, preservatives, glycerin, alcohol, carbonating agents used in carbonated beverages, and the like, and the above-described components may be used independently or in combination. The above-described components may be added in an appropriate amount within a range that does not inhibit the osteoarthritis alleviation effect according to the present invention. For example, the above-described components may be included in an amount of 0.1% to 1% by weight based on the total weight of the composition having the osteoarthritis alleviation effect.

In addition, the composition having the osteoarthritis alleviation effect may include a food additive acceptable from a food science perspective, and may further include an appropriate carrier, excipient, and diluent commonly used in the preparation of a composition having an osteoarthritis alleviation effect.

These immune-boosting compositions in the form of beverages, granules, tablets, powder, pills, and capsules are easy to carry and can be easily taken anytime, anywhere.

Hereinafter, a method of preparing a composition having an osteoarthritis alleviation effect according to the present invention will be described in detail.

The preparation method according to the present invention may include (S1) a step of inoculating Morinda citrifolia fruit with lactic acid bacteria and fermenting and aging it, (S2) a step of preparing an FME from the fermented Morinda citrifolia obtained by the fermentation and aging, and (S3) a step of concentrating the FME.

### Step (S1)

In Step S1, Morinda citrifolia fruit may be inoculated with lactic acid bacteria and fermented and aged.

The lactic acid bacteria may be Lactobacillus plantarum NST1805 (Accession Number: KCCM12833P).

The lactic acid bacteria may be inoculated into Morinda citrifolia fruit in the form of a lactic acid bacteria solution, preferably a lactic acid bacteria aqueous solution. At this time, 0.1 to 0.5 parts by weight of the lactic acid bacteria solution may be inoculated based on 100 parts by weight of the Morinda citrifolia fruit. When the above-described inoculation amount of the lactic acid bacteria solution is satisfied, the above-described bioconversion may occur smoothly, so that fermented Morinda citrifolia containing a component that exhibits a significant effect in osteoarthritis alleviation is obtained.

Meanwhile, the fermentation and aging may be carried out at 35 to 40° C. for 60 days or more. Specifically, the fermentation may be carried out for 60 days or more from the start of fermentation, thereby preventing the formation of mold and homogenizing the fermented liquid.

The fermentation and aging temperature may be 35 to 40° C. When the fermentation and aging temperature is lower than 30° C., fermentation may not occur normally, and when it exceeds 40° C., the fermentation and aging time may be prolonged, which may cause the lactic acid bacteria to die.

### Step (S2)

In Step S2, a concentrated FME may be prepared from the fermented Morinda citrifolia obtained by the fermentation.

First, the fermented Morinda citrifolia obtained through the fermentation may be juiced.

At this time, the fermented Morinda citrifolia that has undergone the fermentation process may include both Morinda citrifolia fruit and fermented liquid. Meanwhile, before the juicing step, a step of preheating the fermented Morinda citrifolia may be further included. The preheating may be performed by heating the fermented Morinda citrifolia at a low temperature, and the preheating may soften the fiber of the fermented Morinda citrifolia, thereby increasing the juice yield and enabling nutrients to be well extracted from the fermented Morinda citrifolia.

At this time, the preheating temperature is preferably 70 to 80° C. When the preheating temperature is lower than 60° C., there is no change in the state of the fiber in the fermented Morinda citrifolia, and thus the effect of improving the juice yield cannot be expected. When the preheating temperature is higher than 80° C., the fermented Morinda citrifolia may be denatured, or the lactic acid bacteria in the fermented Morinda citrifolia may be sterilized.

In particular, when the preheating temperature is 90° C. or higher, there is a problem that more than 90% of the lactic acid bacteria are sterilized. In addition, the preheating time is preferably one to two hours. When the preheating time is less than 30 minutes, there is no change in the state of the fiber in the fermented Morinda citrifolia, so the effect of improving the juice yield cannot be expected. When the preheating time exceeds two hours, the fermented Morinda citrifolia may be denatured, or the lactic acid bacteria in the fermented Morinda citrifolia may die.

The juicing may be performed by juicing the Morinda citrifolia fruit and fermented liquid contained in the fermented Morinda citrifolia to prepare an FME. At this time, the juicing may be performed by applying pressure to the fermented Morinda citrifolia, and the juicing is preferably performed within a preset pressure range. When the juicing pressure is below the preset pressure range, the juicing may not be performed properly, resulting in a decrease in yield, and when it exceeds the preset pressure range, byproducts of the Morinda citrifolia fruit may also be mixed, resulting in a decrease in the purity of the extract.

In addition, after the juicing, a filtration process may further be performed to improve purity and enhance product reliability.

In one embodiment, the filtration of the juice may be performed through an 80 mesh filter.

### Step (S3)

In Step S3, the fermented Morinda citrifolia juice may be concentrated to obtain FME.

After juicing, the moisture contained in the FME may be evaporated to concentrate it to 55 to 65 Brix.

More specifically, the above FME may be concentrated to 60 (±5) Brix at a temperature of 60° to 65° C. to prepare a concentrate. At this time, the Brix value may be measured using a saccharimeter (PAL-3) manufactured by ATAGO.

Thereafter, the FME may be sterilized. The sterilization method is not limited, and any typical sterilization method in the art may be used.

In one embodiment, the FME may be sterilized at 65° C. to 95° C. for 30 minutes.

The FME may contain deacetylasperulosidic acid as an active ingredient, and it may be contained in an amount of 5.76 to 8.64 mg per 1 g of FME.

### EXAMPLES

Morinda citrifolia fruit was washed, frozen at −27° C. to remove bacteria, and thawed at 37° C. for two days. Thereafter, it was fermented with a 2% aqueous solution of Lactobacillus plantarum NST 1805 (Accession No.: KCCM12833P) at 37° C. for 60 days.

The fermented Morinda citrifolia was preheated at 78° C. for 1 hour and 30 minutes and then juiced using a juicing device.

Thereafter, the juiced fermented Morinda citrifolia was filtered using an 80 mesh filter, concentrated to prepared FME of 60 Brix, which was then sterilized at 65° C. to 95° C. for 30 minutes.

### Experimental Example 1: Effect on Nitric Oxide (NO) Production

Excessive amounts of NO are produced at sites of inflammation, promoting necrosis of cell tissue. Therefore, measuring the amount of NO produced at sites of inflammation may reveal anti-inflammatory effects.

To evaluate the osteoarthritis alleviation effect of FME, human chondrosarcoma SW1353 cells (hereinafter referred to as SW1353 cells) were used.

SW1353 cells were treated with FME, and IL-1β, a pro-inflammatory cytokine, was treated to create an environment similar to osteoarthritis. An anti-inflammatory effect was confirmed in comparison with an osteoarthritis-induced test group.

SW1353 cells were treated with FME at various concentrations and treated with 10 ng/mL of IL-1β, and cultured for 24 hours. Thereafter, 100μL of the supernatant was mixed with an equal volume of Griess reagent, and the resulting mixture was allowed to react at room temperature for 10 minutes. The absorbance was measured at 540 nm using an enzyme-linked immunosorbent assay (ELISA) reader.

The lower the absorbance, the lower the NO production, and the lower the NO production, the better the anti-inflammatory effect.

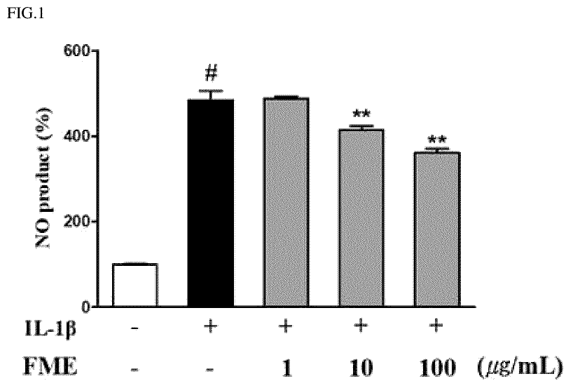

Referring to FIG. 1, it was confirmed that the amount of NO production increased when IL-1β was treated, while the amount of NO production decreased in the test group co-treated with FME. These results indicate that the FME significantly inhibited NO production.

### Experimental Example 2. Inhibitory Effect of FME on Cartilage Degradation- and Inflammation-Related Protein Expression

Collagen and gelatin are main proteins that constitute cartilage, and matrix metalloproteinases (MMPs) are known to promote cartilage destruction and induce osteoarthritis when an inflammatory response is induced in the joints. This experimental example was performed to confirm the inhibitory effect of FME when the protein expression of MMP −1, −3, and −13 is increased in an osteoarthritis-induced cell model.

In the experimental method, SW1353 cells were plated in a 6-well plate at 5.0×10⁵ cells per well and cultured in a CO₂ incubator for 24 hours. FME was pretreated at various concentrations, IL-1β (10 ng/mL) was treated, and the cells were cultured for 24 hours. After washing twice with phosphate-buffered saline (PBS), the cells were lysed with a radio-immunoprecipitation assay (RIPA) buffer (Rockland Inc., Gilbertsville, PA, USA) containing a protease inhibitor cocktail and ethylenediaminetetraacetic acid (EDTA) solution and then allowed to stand at 4° C. for 10 minutes.

Cell lysates were centrifuged at 4° C. and 15,000 rpm for 15 minutes, and protein was quantified in the supernatant using Bradford reagent (Bio-Rad, Hercules, CA, USA). Samples were separated by electrophoresis on a 10% polyacrylamide gel, transferred to a polyvinylidene difluoride membrane, and blocked (5% blocking reagent in tris-buffered saline with Tween-20 (TBST)) for one hour at room temperature.

The samples were treated with a primary antibody overnight at 4° C., washed three times with 0.1% TBST, and then allowed to react with a secondary antibody for one hour. After the samples were washed with 0.1% TBST and allowed to react with the enhanced chemiluminescence western blotting detection kit (Bio-Rad), protein bands were identified using ChemiDoc (Bio-Rad) equipment, and the band density was measured using Image J software.

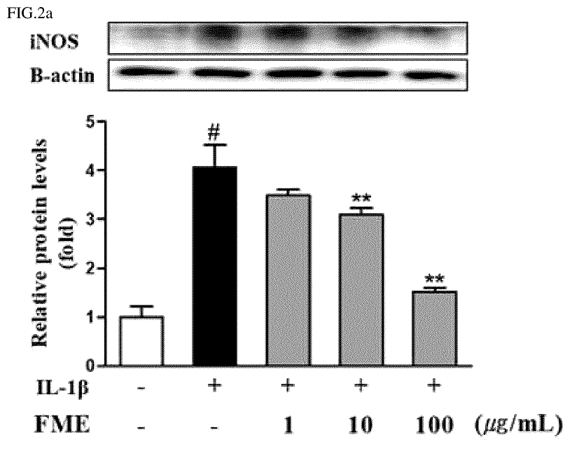

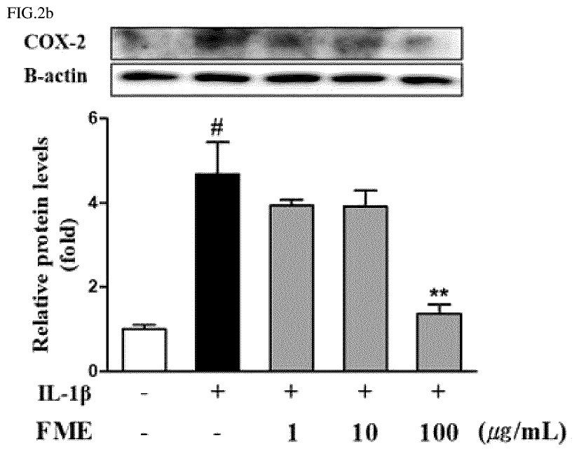

Referring to FIGS. 2A and 2B, the effect of FME on iNOS and COX-2 expression was confirmed in an osteoarthritis-induced cell model in which iNOS and COX-2 expression was increased by IL-1β treatment that induced an inflammatory response, and inhibition of this expression was observed when FME was treated. iNOS showed statistical significance from 10 ng/mL, and inhibition of COX-2 expression showed statistical significance at 100μg/mL treatment.

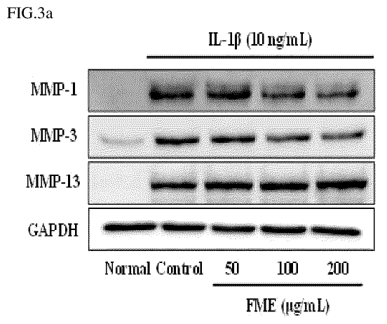

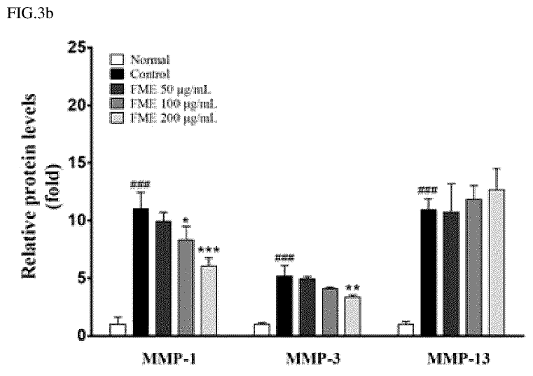

Referring to FIGS. 3A and 3B, it was confirmed that when the osteoarthritis-induced cell model was treated with IL-1β, the protein expression of MMP −1, −3, and −13 increased, and in the test group treated in combination with FME, the expression of MMP −1 and −3 significantly decreased.

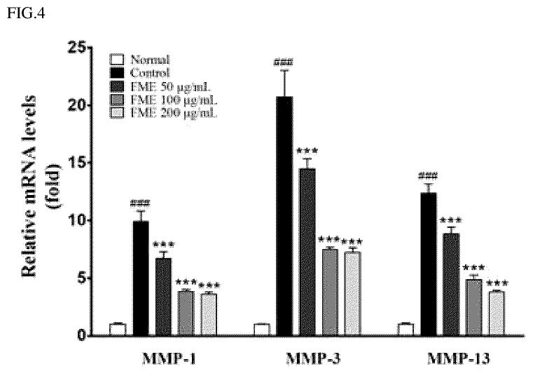

Referring to FIG. 4, it was confirmed that when the osteoarthritis-induced cell model was treated with IL-1β, the mRNA expression of MMP−1, −3, and −13 increased, and in the test group treated in combination with FME, the mRNA expression of MMP −1, −3, and −13 significantly decreased.

### Experimental Example 3. Inhibitory Effect of FME on Cartilage Degradation- and Inflammation-Related mRNA Expression

When osteoarthritis is induced, chondrocytes are stimulated by pro-inflammatory cytokines, and the expression of MMPs and a disintegrin and metalloproteinase with thrombospondin motifs (ADAMTS) −4 and −5, which degrade collagen and proteoglycan, which are proteins that constitute the extracellular matrix (ECM) of cartilage, is promoted, and the expression of inflammatory cytokines also increases. This experimental example was performed to confirm the inhibitory effect of FME when the expression of MMP −1, −3, and −13 and ADAMTS −4 and −5, which are proteins that degrade the ECM, and the expression of inflammatory cytokines (TNF-α, IL-6) increased in an osteoarthritis-induced cell model.

In the experimental method, SW1353 cells were plated in a 6-well plate at 5.0×10⁵ cells per well and cultured in a CO₂ incubator for 24 hours. FME was pretreated at various concentrations, and cells were treated with IL-1β (10 ng/mL) and cultured for 24 hours. Total RNA was obtained using Nucleozol (Macherey-Nagel, Germany), and after quantifying the purified RNA, one-step real-time PCR was performed using RNA-direct™ SYBR® Green Realtime PCR Master Mix (QRT-201, TOYOBO, Osaka, Japan).

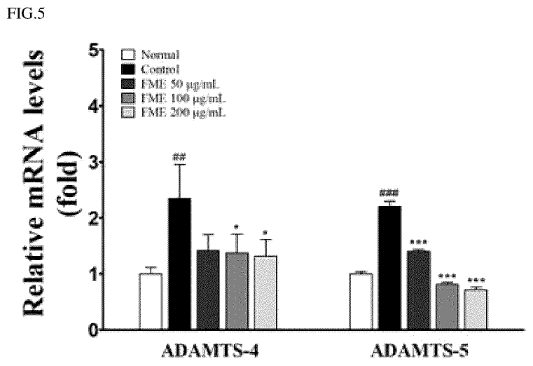

Referring to FIG. 5, when SW1353 chondrosarcoma cells were treated with IL-1β, the expression of ADAMTS −4 and −5, which are enzymes that degrade ECM, increased, and in the test group treated in combination with FME, the mRNA expression of ADAMTS −4 and −5 significantly decreased.

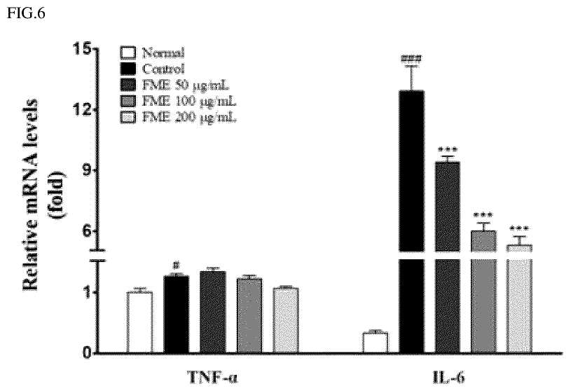

Referring to FIG. 6, when SW1353 chondrosarcoma cells were treated with IL-1β, mRNA expression of inflammatory cytokines (TNF-α, IL-6) increased. It was confirmed that mRNA expression of IL-6 was inhibited in the test group treated in combination with FME.

### Experimental Example 4. Mmp-9 Inhibitory Effect of FME

In the experimental method, SW1353 cells were plated in a 6-well plate at 5.0×10⁵ cells per well and cultured in a CO₂ incubator for 24 hours. Cells were pretreated with various concentrations of FME, treated with IL-1β (10 ng/mL), and then cultured for 24 hours. After culture, the expression level of MMP-9 was measured using an ELISA Kit using the cell supernatant.

The expression level of MMP-9 in a group treated with an unfermented Morinda citrifolia extract (ME) was measured by the same method.

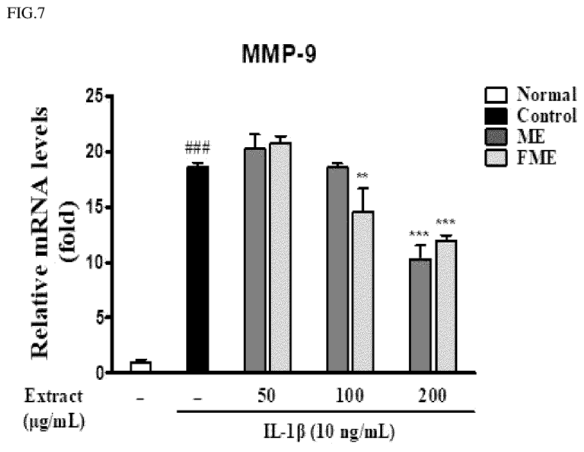

Referring to FIG. 7, the IL-1β treatment group showed a significant increase in MMP-9 expression compared to the normal group, and it was confirmed that MMP-9 expression was decreased by ME treatment and FME treatment. In addition, among the unfermented ME treatment groups, the 200μg/mL treatment group showed a significant decrease compared to the osteoarthritis-induced group (control group), but among the FME treatment groups, the 100 and 200μg/mL treatment groups showed a significant decrease compared to the osteoarthritis-induced group (control group). These results confirmed that FME effectively inhibited the mRNA expression of MMP-9 even at relatively low concentrations.

While the embodiments of the present invention have been described above with reference to the attached drawings, those skilled in the art will understand that the present invention can be implemented in other specific forms without altering the technical concept or essential features thereof. Therefore, the above-described embodiments should be understood to be illustrative in all respects and not restrictive.

Modes for carrying out the invention were described above in the Modes of the Invention.

### Industrial Applicability

The present invention can provide a composition including a fermented Morinda citrifolia extract (FME) having an osteoarthritis alleviation effect and a method of preparing a composition including FME.

### Original Deposit of Biological Material

Biological Material: Lactobacillus plantarum NST1805

Depositor: Korean Culture Center of Microorganisms (KCCM), having an address at Yurim B/D, 45, Hongjenae-2ga-gil, Seodaemun-gu, SEOUL 03641, Republic of Korea.

Accession Number: KCCM12833P

Date of acceptance: Nov. 17, 2020

Under the conditions of the Budapest Treaty, access to this deposit will be available during the pendency of the application to the Commissioner of Patents and Trademarks and persons determined by the Commissioner to be entitled thereto upon request. Upon allowance of any claims in the application, the Applicant(s) will make available to the public, pursuant to 37 C.F. R. § 1.808, sample(s) of the deposit. This deposit of the microorganism will be maintained for the enforceable life of the patent and will be replaced if it becomes nonviable during that period. Additionally, Applicant(s) have satisfied all the requirements of 37 C.F.R. § § 1.801-1.809, including providing an indication of the viability of the sample upon deposit. Applicant(s) have no authority to waive any restrictions imposed by law on the transfer of biological material or its transportation in commerce. Applicant(s) do not waive any infringement of their rights granted under this patent. Unauthorized microorganism multiplication is prohibited. The microorganism may be regulated.

1. A composition comprising a fermented Morinda citrifolia extract having an osteoarthritis alleviation effect.

2. The composition of claim 1, wherein the fermented Morinda citrifolia extract contains deacetylasperulosidic acid as an active ingredient.

3. The composition of claim 2, wherein the active ingredient is contained in an amount of 5.76 to 8.64 mg per 1 g of the fermented Morinda citrifolia extract.

4. The composition of claim 3, wherein the fermented Morinda citrifolia extract is prepared by inoculating Morinda citrifolia with Lactobacillus plantarum NST1805 (Accession Number: KCCM12833P) and then fermenting the Morinda citrifolia.

5. The composition of claim 4, wherein the fermented Morinda citrifolia extract is prepared by juicing the fermented Morinda citrifolia and concentrating the juice to 55 to 65 Brix.

6. A food composition having an osteoarthritis alleviation effect, comprising the fermented Morinda citrifolia extract according to claim 1.

7. A method of preparing a composition including a fermented Morinda citrifolia extract having an osteoarthritis alleviation effect, comprising:
inoculating Morinda citrifolia with lactic acid bacteria and fermenting the Morinda citrifolia;
preparing a fermented Morinda citrifolia extract from the fermented Morinda citrifolia obtained by fermentation; and
concentrating the fermented Morinda citrifolia extract.

8. The method of claim 7, wherein the lactic acid bacterium is Lactobacillus plantarum NST1805 (Accession Number: KCCM12833P).
